(19) United States
(12) Patent Application Publication
(10) Pub. No.: US 2017/0290700 A1
(43) Pub. Date: Oct. 12, 2017

(54) FOOT WARMING ASSEMBLY

(71) Applicant: Merline Guillaume, Irvington, NJ (US)

(72) Inventor: Merline Guillaume, Irvington, NJ (US)

(21) Appl. No.: 15/093,903

(22) Filed: Apr. 8, 2016

(51) Int. Cl. A61F 7/02; A61F 7/00

(52) U.S. Cl. CPC A61F 7/02; A61F 7/007; A61F 2007/0045; A61F 2007/0047; A61F 2007/0078; A61F 2007/0088; A61F 2007/0204; A61F 2007/0268

(57) ABSTRACT

A foot warming assembly to receive and heat a person's feet has a housing that consists of a bottom wall and a perimeter wall. The perimeter wall is attached to the bottom wall and extends upwards to define an upper edge. A dividing wall is attached to the perimeter wall between the bottom wall and the upper edge and divides the area inside the housing into a compartment below and an upper portion above the dividing wall. A heating element is positioned within the compartment. A controller is electrically coupled to the heating element to turn the heating element on or off. A power supply is in electrical communication with the controller and the heating element to supply electrical power to the heating element. When the controller is turned on, the heating element warms the sand in the upper portion and the sand warms a person's feet.

### BACKGROUND OF THE DISCLOSURE

### Field of the Disclosure

The disclosure relates to foot warming devices and more particularly pertains to a new foot warming device that warms a person's feet by warming up sand into which their feet will be placed.

### SUMMARY OF THE DISCLOSURE

An embodiment of the disclosure meets the needs presented above by generally comprising a housing that has a bottom wall and a perimeter wall extending upwardly from the bottom wall. The perimeter wall has an upper edge that defines an opening into the housing. A dividing wall is attached to the perimeter wall and is spaced from the bottom wall and the upper edge. A compartment is defined in the housing positioned between the dividing wall and the bottom wall. An upper portion is defined in the housing between the upper edge of the housing and the dividing wall. The upper portion is configured to receive sand. A heating element is positioned within the compartment to heat the sand in the upper portion. A controller is electrically coupled to the heating element. It may be set to an on position to turn on the heating element or an off position to turn off the heating element. A power supply is in electrical communication with the controller and the heating element to supply electrical power to the heating element when the controller is placed in the on position.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a rear isometric view of a foot warming assembly according to an embodiment of the disclosure.

FIG. 2 is a front view of an embodiment of the disclosure.

FIG. 3 is a top cross-sectional view of an embodiment of the disclosure.

FIG. 4 is a front cross-sectional view of an embodiment of the disclosure.

FIG. 5 is a top view of an embodiment of the disclosure.

### DESCRIPTION OF THE PREFERRED EMBODIMENT

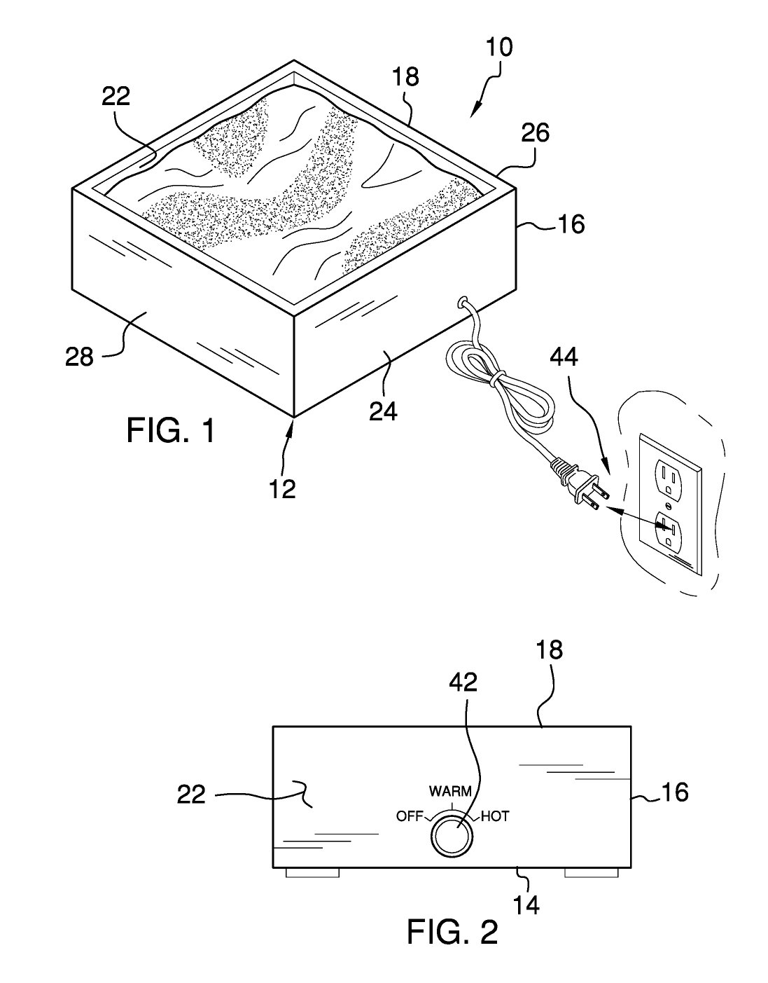

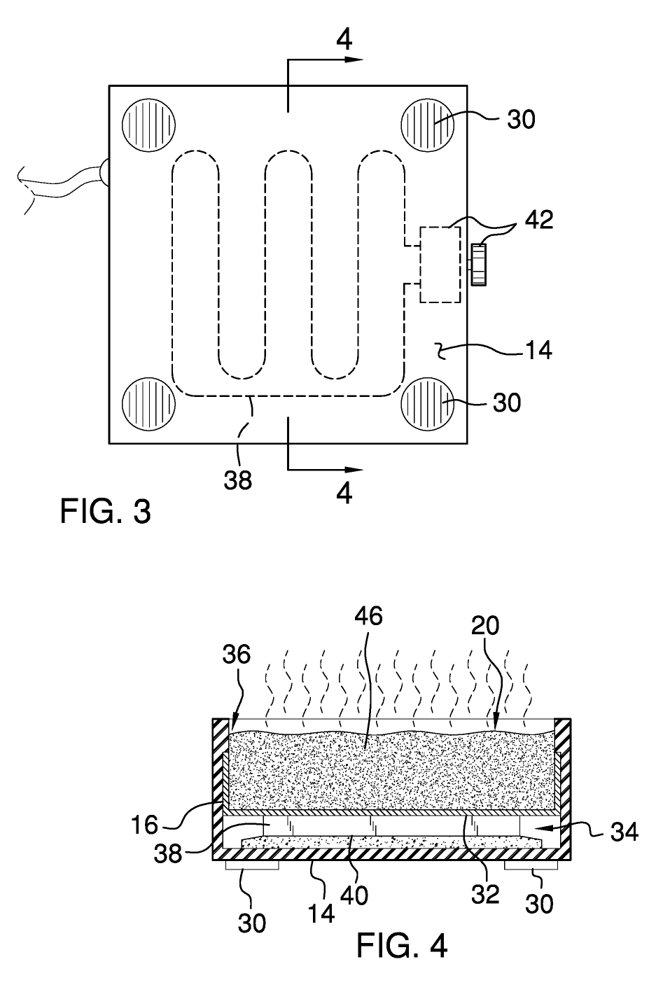

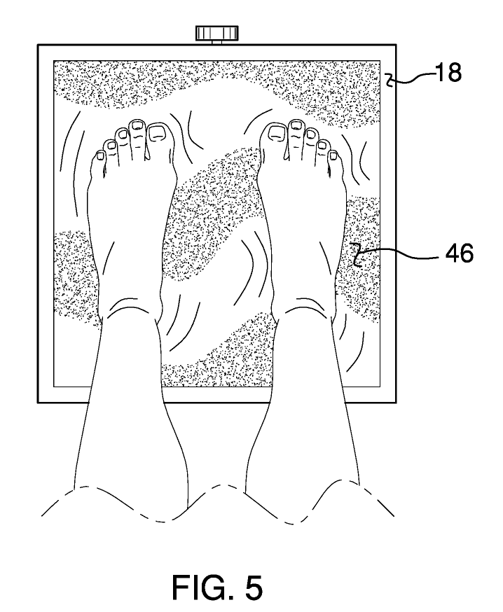

With reference now to the drawings, and in particular to FIGS. 1 through 5 thereof, a new foot warming device embodying the principles and concepts of an embodiment of the disclosure and generally designated by the reference numeral 10 will be described.

As best illustrated in FIGS. 1 through 5, the foot warming assembly 10 generally comprises a housing 12 that consists of a bottom wall 14 and a perimeter wall 16 that is attached to the bottom wall 14 and extending upwardly therefrom. The perimeter wall 16 has an upper edge 18 defining an opening 20 into the housing 12. The perimeter wall 16 has a front wall 22, a rear wall 24, a first lateral wall 26 and a second lateral wall 28. A plurality of non-skid members 30 may be attached to bottom surface of the bottom wall 14 and extending downwardly from the bottom wall 14 of the housing 12.

A dividing wall 32 is attached to the perimeter wall 16 and is separated from the bottom wall 14 and the upper edge 18 of the perimeter wall 16. The dividing wall 32 defines a compartment 34 in the housing 12 positioned below the dividing wall 32 and between the bottom wall 14 and the dividing wall 32. The dividing wall 32 also defines an upper portion 36 in the housing 12 positioned above the dividing wall 32 and between the dividing wall 32 and the upper edge 18 of the perimeter wall 16. The upper portion 36 of the housing 12 is configured to receive sand.

A heating element 38 is positioned within the compartment 34 and is between the bottom wall 14 and the dividing wall 32. An insulating layer 40 may be positioned between the heating element 38 to thermally insulate the heating element 38 from the bottom wall 14. The heating element 38 may comprise a conventional electrically heated wire or cord. However, the heating element 38 may comprise tubing with fluid that is heated or air heated and blown through the housing 12. A controller 42 is mounted on the housing 12 and is electrically coupled to the heating element 34. The controller 42 may be actuated to an on position or an off position. Further, the on position may allow for adjustable levels of heat output such as a low setting and a high setting. A power supply 44 is in electrical communication with the controller 42 and the heating element 38 to supply electrical power to the heating element 38 when the controller 42 is placed in the on position. The power supply 44 may include, as shown in FIG. 1, a power cord pluggable into an electrical outlet. Alternatively, the power supply 44 may comprise a battery.

In use, a person may fill the upper portion 36 of the housing 12 with sand 46. The sand 46 is heated by the heating element 38 to the adjustable level set by the controller 42. The person may then place their feet on the sand to warm the bottom of their feet or bury their feet under the sand 46 to warm the whole surface of the feet and to relax the person.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A foot warming assembly configured for receiving and heating a person's feet, said foot warming assembly comprising:
a housing having a bottom wall and a perimeter wall being attached to said bottom wall and extending upwardly therefrom, said perimeter wall having an upper edge defining an opening into said housing;
a dividing wall being attached to said perimeter wall and being spaced from said bottom wall and said upper edge to define a compartment between said bottom housing and said dividing wall and an upper portion between said dividing wall and said upper edge of said housing, said upper portion being configured to receive sand;
a heating element being positioned within said compartment and being between said bottom wall and said dividing wall;
a controller being mounted on said housing and being electrically coupled to said heating element, said controller being set to an on position to turn on said heating element or an off position to turn off said heating element;
a power supply being in electrical communication with said controller and said heating element to supply electrical power to said heating element when said controller is placed in said on position; and
wherein said heating element is configured to heat sand in said upper portion of said housing to warm a person's feet.

2. The foot warming assembly according to claim 1, wherein said perimeter wall has a front wall, a rear wall, a first lateral wall and a second lateral wall.

3. The foot warming assembly according to claim 1, further including an insulating layer being positioned between said heating element and said bottom wall of said housing.

4. The foot warming assembly according to claim 1, further including a plurality of non-skid members being attached to bottom surface of said bottom wall and extending downwardly from said bottom wall of said housing.

5. A foot warming assembly configured for receiving and heating a person's feet, said foot warming assembly comprising:
a housing having a bottom wall and a perimeter wall being attached to said bottom wall and extending upwardly therefrom, said perimeter wall having an upper edge defining an opening into said housing, said perimeter wall having front wall, rear wall, a first lateral wall and a second lateral wall;
a dividing wall being attached to said perimeter wall and being spaced from said bottom wall and said upper edge to define:
a compartment in said housing positioned below said dividing wall and between said bottom wall and said dividing wall;
an upper portion in said housing positioned above said dividing wall and between said dividing wall and said upper edge, said upper portion being configured to receive sand;
a heating element being positioned within said compartment and being between said bottom wall and said dividing wall;
a controller being mounted on said housing and being electrically coupled to said heating element, said controller being set to an on position to turn on said heating element or an off position to turn off said heating element;
a power supply being in electrical communication with said controller and said heating element to supply electrical power to said heating element when said controller is placed in said on position;
an insulating layer being positioned between said heating element and said bottom wall of said housing;
a plurality of non-skid members being attached to bottom surface of said bottom wall and extending downwardly from said bottom wall of said housing.
